(19) United States
(12) Patent Application Publication
(10) Pub. No.: US 2021/0085189 A1
(43) Pub. Date: Mar. 25, 2021

(54) BLOOD PRESSURE MANAGEMENT DEVICE, BLOOD PRESSURE MANAGEMENT METHOD, AND NON-TRANSITORY COMPUTER-READABLE STORAGE MEDIUM STORING BLOOD PRESSURE MANAGEMENT PROGRAM

(71) Applicant: OMRON HEALTHCARE Co., Ltd., Kyoto (JP)

(72) Inventors: Hironori Sato, Kyoto (JP); Sirui Yeap, Kyoto (JP)

(21) Appl. No.: 17/100,493

(22) Filed: Nov. 20, 2020

Related U.S. Application Data
(63) Continuation of application No. PCT/JP2019/020054, filed on May 21, 2019.

(30) Foreign Application Priority Data
May 24, 2018 (JP) 2018-099810

(51) Int. Cl. A61B 5/022; A61B 5/00

(52) U.S. Cl. CPC A61B 5/022; A61B 5/742

(57) ABSTRACT

A blood pressure management device includes an acquisition unit that acquires a measurement value of blood pressure, a setting unit that sets, based on blood pressure fluctuation characteristics, a unit measurement period including a plurality of opportunities, and a calculation unit that, when a second measurement is performed in a different opportunity from a first measurement within the unit measurement period, calculates a representative value for the unit measurement period by using both a first measurement value to and a second measurement value.

### CROSS-REFERENCE TO RELATED APPLICATIONS

This application is the U.S. national stage application filed pursuant to 35 U.S.C. 365(c) and 120 as a continuation of International Patent Application No. PCT/JP2019/020054, filed May 21, 2019, which application claims priority to from Japanese Patent Application No. 2018-099810, filed May 24, 2018, which application are incorporated herein by reference in their entireties.

### TECHNICAL FIELD

The present invention relates to a blood pressure management device, a blood pressure management method, and a non-transitory computer-readable storage medium storing a blood pressure management program configured to calculate a representative blood pressure value from a plurality of blood pressure values.

### BACKGROUND ART

Patent Document 1 discloses a blood pressure measurement device that acquires a plurality of measurement values from continuous blood pressure measurements, and calculates a representative value of the measurement values. In this blood pressure measurement device, of the acquired measurement values, the measurement values measured in the same opportunity (e.g., over 10 minutes) are extracted. Then, an average value of the extracted measurement values is displayed as the representative blood pressure value.

### CITATION LIST

### Patent Literature

Patent Document 1: JP 2013-165812 A

### SUMMARY OF INVENTION

### Technical Problem

Blood pressure varies depending on measurement time, measurement environment, measurement conditions, and the like. For example, a measurement value obtained from a measurement performed after waking up is useful for accurately determining the physical condition and the like of the subject. Thus, there is a demand for appropriately reflecting useful measurement values such as the measurement value obtained after waking up in the representative blood pressure value.

For example, in the blood pressure measurement device disclosed in Patent Document 1, upon calculating the representative value in a specific time period, when the second measurement is performed at a different opportunity from the first measurement, the second measurement is not reflected in the representative blood pressure value.

The present invention has been conceived in light of the circumstances described above, and an object of the present invention is to provide a blood pressure management device, a blood pressure management method, and a non-transitory computer-readable storage medium storing a blood pressure management program that are capable of calculating a representative blood pressure value in which useful measurement results are appropriately reflected.

### Solution to Problem

The present invention adopts the following configurations in order to achieve the above-mentioned object.

Specifically, a blood pressure management device according to an aspect of the present disclosure includes an acquisition unit configured to acquire a measurement value obtained by measurement of blood pressure, a setting unit configured to set, based on blood pressure fluctuation characteristics, a unit measurement period including a plurality of opportunities, and a calculation unit configured to calculate, based on the measurement value, a representative value of the blood pressure for the unit measurement period. When an opportunity in which a second measurement is performed and an opportunity in which a first measurement is performed are different within the unit measurement period, the calculation unit calculates the representative value for the unit measurement period by using both a first measurement value obtained by the first measurement and a second measurement value obtained by the second measurement.

In one opportunity, a series of measurement actions including at least one measurement are performed. In the one opportunity, a series of actions are performed, for example, from starting preparations for blood pressure measurement until completing a plurality of consecutive blood pressure measurements. The measurements performed in the same opportunity are, for example, measurements performed within 10 minutes. Further, the representative value of the blood pressure is calculated by using a plurality of measurement values, and is an average value, a median value, or the like of the plurality of measurement values.

According to the configuration described above, for example, in a case in which the second measurement is performed in the specific unit measurement period, even when the second measurement is performed in a different opportunity from the first measurement, the representative value of the blood pressure is calculated by using the first measurement result and the second measurement result. Thus, even when the second measurement is performed in a different opportunity from the first measurement, a representative value of the blood pressure can be obtained, where the representative value satisfies a prescribed number and in which useful measurement results are appropriately reflected.

In the blood pressure management device according to the aspect described above, the calculation unit determines whether the opportunity in which the first measurement is performed and the opportunity in which the second measurement is performed are identical based on a representative time period required for a measurement action of one opportunity.

In the blood pressure management device according to the aspect described above, when the first measurement and the second measurement have been performed within a unit measurement period of not more than 10 minutes, the calculation unit determines that the opportunity in which the first measurement is performed and the opportunity in which the second measurement is performed are identical.

In the blood pressure management device according to the aspect described above, when the opportunity in which the first measurement is performed and the opportunity in which the second measurement is performed are identical within the unit measurement period, and a third measurement is performed in a different opportunity from the opportunity in which the first measurement is performed within the unit measurement period, the calculation unit calculates the representative value for the unit measurement period by using only the first measurement value and the second measurement value.

In the blood pressure management device according to the aspect described above, when the opportunity in which the first measurement is performed and the opportunity in which the second measurement is performed are different within the unit measurement period, and a third measurement is performed in a different opportunity from the opportunity in which the second measurement is performed within the unit measurement period, the calculation unit calculates the representative value by using only the first measurement value and the second measurement value.

In the blood pressure management device according to the aspect described above, when the opportunity in which the first measurement is performed and the opportunity in which the second measurement is performed are different within the unit measurement period, and a third measurement is performed in an opportunity identical to the opportunity in which the second measurement is performed within the unit measurement period, the calculation unit calculates the representative value by using the first measurement value, the second measurement value, and a third measurement value obtained by the third measurement.

In the blood pressure management device according to the aspect described above, the calculation unit does not use measurement values obtained by fourth and subsequent measurements within the unit measurement period for the calculation of the representative value.

According to the configuration described above, when four or more measurements are performed in the specific unit measurement period, measurement results obtained by the fourth and subsequent measurements are not reflected in the representative value. As a result, the representative value of the blood pressure can be efficiently calculated.

In the blood pressure management device according to the aspect described above, the setting unit sets a plurality of the unit measurement periods to divide one day into the plurality of unit measurement periods.

According to the configuration described above, there is no time of the day (24 hours from a specific time on a chosen day until the same time on the next day) that does not correspond to any of the unit measurement periods. In other words, all times of the day are included in one of the unit measurement periods. Thus, whenever the measurement is performed, a measurement result thereof is reflected in the representative value of the unit measurement period corresponding to the representative value. Therefore, even for subjects having various lifestyles, the representative value of the blood pressure can be calculated in which useful measurement results are appropriately reflected.

In the blood pressure management device according to the aspect described above, the setting unit sets an end time of the unit measurement period based on a measurement time of the first measurement.

According to the configuration described above, by setting the unit measurement period based on measurement information, even when the lifestyle of the subject is not constant, a representative value of the blood pressure can be calculated in which useful measurement results, such as a measurement value obtained when waking up, are appropriately reflected.

In the blood pressure management device according to the aspect described above, the setting unit sets a time period of not more than 15 hours as the unit measurement period.

In the blood pressure management device according to the aspect described above, the acquisition unit further acquires a wake-up time, and the setting unit sets the wake-up time as a start time of the unit measurement period, and based on the wake-up time, sets an end time of the unit measurement period.

According to the configuration described above, by setting the unit measurement period based on the wake-up time, even when the lifestyle of the subject is not constant, useful measurement results, such as the measurement value obtained after waking up, can be easily identified. As a result, the representative value of the blood pressure can be calculated in which useful measurement results, such as the measurement value obtained after waking up, are more appropriately reflected.

In the blood pressure management device according to the aspect described above, in the calculation of the representative value by the calculation unit, a maximum time from acquisition of the first measurement value until an end of a stand-by state for acquisition of the second measurement value is longer than a maximum time from the acquisition of the second measurement value until an end of a stand-by state for acquisition of a third measurement value obtained by a third measurement within the unit measurement period.

### Advantageous Effects of Invention

According to the present invention, it is possible to provide a blood pressure management device, a blood pressure management method, and a non-transitory computer-readable storage medium storing a blood pressure management program that are capable of calculating a representative blood pressure value in which useful measurement results are appropriately reflected.

### BRIEF DESCRIPTION OF DRAWINGS

FIG. 1 is a block diagram illustrating a functional configuration of a blood pressure management device according to an application example.

FIG. 2 is a schematic diagram illustrating an example of a configuration of a blood pressure processing system including a blood pressure management device according to a first embodiment.

FIG. 3 is a block diagram illustrating an example of a hardware configuration of the blood pressure measurement device according to the first embodiment.

FIG. 4 is a block diagram illustrating an example of a hardware configuration of a portable terminal according to the first embodiment.

FIG. 5 is a block diagram illustrating an example of a hardware configuration of a physician terminal according to the first embodiment.

FIG. 6 is a block diagram illustrating an example of a hardware configuration of a server according to the first embodiment.

FIG. 7 is a block diagram illustrating an example of a functional configuration to of a blood pressure processing circuit according to the first embodiment.

FIG. 8 is a state transition diagram illustrating an example of a procedure for representative value calculation processing performed in the blood pressure management device according to the first embodiment.

FIG. 9 is a diagram illustrating examples of measurement values reflected in calculation of a representative value in the representative value calculation processing performed in the blood pressure management device according to the first embodiment.

FIG. 10 is a state transition diagram illustrating an example of the procedure for the representative value calculation processing performed in the blood pressure management device according to a first modified example of the first embodiment.

FIG. 11 is a state transition diagram illustrating an example of the procedure for the representative value calculation processing performed in the blood pressure management device according to a second modified example of the first embodiment.

FIG. 12 is a state transition diagram illustrating an example of the procedure for the representative value calculation processing performed in the blood pressure management device according to a third modified example of the first embodiment.

FIG. 13 is a state transition diagram illustrating an example of the procedure for the representative value calculation processing performed in the blood pressure management device according to a second embodiment.

FIG. 14 is a flowchart illustrating an example of the procedure for the representative value calculation processing performed in a blood pressure processing circuit according to a third embodiment.

### DESCRIPTION OF EMBODIMENTS

Now, with reference to the drawings, embodiments are described. Note that, in the following description, constituent elements having the same function and configuration are denoted with a shared reference symbol. Further, when a plurality of constituent elements having a shared reference symbol are distinguished from one another, distinguishment is made by adding additional symbols following the shared reference symbol. Note that, when there is no particular need in distinguishing a plurality of constituent components, the plurality of constituent components are denoted only with a shared reference symbol without an additional symbol.

### 1. Application Example

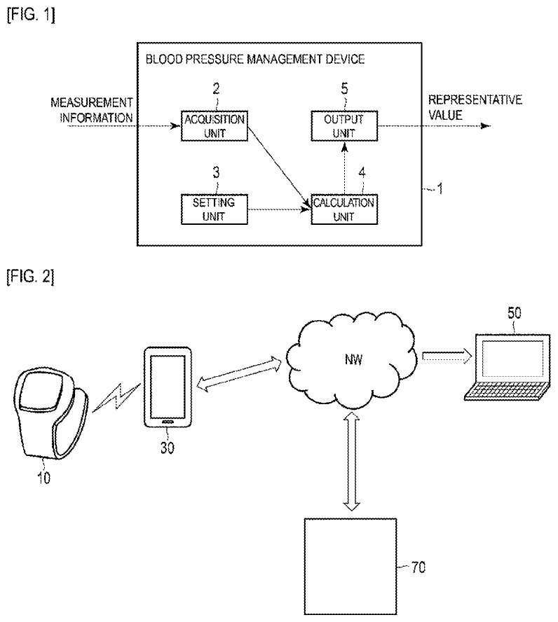

First, with reference to FIG. 1, an example of a blood pressure management device to which the present invention is applied is described.

As illustrated in FIG. 1, a blood pressure management device 1 includes an acquisition unit 2, a setting unit 3, a calculation unit 4, and an output unit 5. The blood pressure management device 1 performs representative value calculation processing based on a blood pressure processing program. The blood pressure processing program is an example of a blood pressure management program.

The acquisition unit 2 acquires blood pressure measurement information with respect to a specific subject to be measured. The measurement information includes a measurement value obtained by measuring blood pressure using a sensor. The blood pressure measurement value is associated with a measurement date/time (a measurement date and a measurement time). Thus, the measurement information can include the measurement value and the measurement date/time of blood pressure.

The setting unit 3 sets a unit measurement period based on fluctuation characteristics of the blood pressure. The unit measurement period includes a plurality of opportunities at which at least one measurement is performed. The unit measurement period is, for example, set times relating to a time period, switching times of the time period, or the like. The set times relating to the time to period include, for example, a start time and an end time of the time period.

The calculation unit 4 acquires the blood pressure measurement information from the acquisition unit 2, and acquires the unit measurement period from the setting unit 3. The calculation unit 4 calculates a representative blood pressure value for each of the unit measurement periods, based on the blood pressure measurement value and the unit measurement period. The representative blood pressure value in a specific unit measurement period is calculated, for example, using at least one of the measurement values obtained in the specific unit measurement period. The representative blood pressure value for the unit measurement period is, for example, an average value or a median value of two or more measurement values measured within the specific unit measurement period. The output unit 5 outputs the calculated representative value to an external device.

In the specific unit measurement period, when two or three measurements including a first measurement are performed within 10 minutes, the calculation unit 4 uses measurement results of the measurements performed within the 10 minutes to calculate the representative blood pressure value. After the representative blood pressure value has been calculated, even when an additional measurement is performed in the specific unit measurement period, the calculation unit 4 does not reflect the measurement result obtained by the measurement performed after the representative blood pressure value has been calculated in the representative blood pressure value.

Here, “10 minutes” is an example of “a representative time period required for a measurement action of one opportunity.” One opportunity indicates a series of measurement actions including at least one measurement. In one opportunity, a series of actions are performed, for example, from starting preparations for blood pressure measurement until completing a plurality of consecutive blood pressure measurements.

Further, the “two or three” blood pressure measurements are an example of “a prescribed number of measurements for calculating a representative blood pressure value.” The prescribed number of measurements is a number of blood pressure measurements recommended to be used for the calculation of the representative value. Guidelines and the like regarding blood pressure measurement stipulate the prescribed number of measurements, on the basis of blood pressure fluctuation characteristics and the like. The prescribed number of measurements is, for example, “once”, “twice”, or “two or three times”. When the prescribed number of measurements is “two or three times”, “two” is the minimum prescribed number and “three” is the maximum prescribed number. Further, when calculating the representative value, a measurement result obtained by a measurement performed within the time period of one opportunity is preferably used.

Thus, with a configuration such as that described above, it is possible to calculate the representative blood pressure value in which a number of measurement values satisfying the prescribed number is reflected.

Further, in the specific unit measurement period, even when the second measurement is performed 10 minutes or more after the first measurement, the calculation unit 4 calculates the representative blood pressure value using the measurement result of the first measurement and the measurement result of the second measurement.

Here, “when the second measurement is performed 10 minutes or more after the first measurement” is an example of “when a first measurement and a second measurement are not performed within a time period of one opportunity.” It is known that even if the first measurement and the second measurement are not performed within the time period of one opportunity, the measurement result of the first measurement and the measurement result of to the second measurement are useful as values used for the measurement of the representative value.

Thus, with a configuration such as that described above, even when the first measurement and the second measurement are not performed within the time period of one opportunity, the representative value is reliably calculated using useful measurement results. As a result, it is possible to calculate the representative blood pressure value in which a number of measurement values satisfying the prescribed number is reflected, and in which useful blood pressure values are appropriately reflected.

### 2. First Embodiment

A first embodiment of a blood pressure management device according to the application example described above is described below. In the following, a blood pressure processing system is described that includes a blood pressure measurement device provided with a blood pressure processing circuit as an example of the blood pressure management device.

### 2.1 Overall Configuration Example

FIG. 2 is a diagram schematically illustrating an example of an application scene of the blood pressure processing system according to the present embodiment. The blood pressure processing system according to the present embodiment is a system that calculates the representative blood pressure value for each of the unit measurement periods using the blood pressure values measured from the subject, and that stores or presents the calculated representative value.

The blood pressure varies depending on various factors such as time, measurement environment, and measurement conditions. A correct blood pressure measurement method is stipulated by guidelines and the like of each country, on the basis of blood pressure fluctuation characteristics.

It is assumed that the blood pressure processing system according to the present embodiment is used in a region in which using two or three measurement results to calculate the representative value in a specific unit measurement period is recommended. It is assumed that the blood pressure processing system according to the present embodiment is used in, for example, the United States. For example, in United States guidelines for the management of hypertension, due to blood pressure fluctuation characteristics and the like, it is recommended to measure at two or more opportunities per day and to perform two or more blood pressure measurements at each opportunity. For example, it is preferable to perform a blood pressure measurement within one hour after waking up and within one hour before going to bed.

As illustrated in FIG. 2, the blood pressure processing system includes a blood pressure measurement device 10 and a portable terminal 30. The blood pressure measurement device 10 and the portable terminal 30 are connected via near-field wireless communication or wired communication. The blood pressure processing system may further include a physician terminal 50 and a server 70. In this case, the portable terminal 30 is connected to each of the physician terminal 50 and the server 70 via a network NW. In this way, the blood pressure measurement device 10 can be connected to each of the physician terminal 50 and the server 70 via the portable terminal 30. In other words, the blood pressure measurement device 10 can communicate with each of the physician terminal 50 and the server 70 via the portable terminal 30. In the present embodiment, communication between the portable terminal 30, the physician terminal 50, and the server 70 can be achieved by communication via the network NW, for example. However, the present embodiment is not limited to this example, and the communication between the portable terminal 30, the physician terminal 50, and the server 70 may be achieved by near-field wireless communication or the wired communication.

The blood pressure measurement device 10 is a wearable device that is worn at a freely-selected measurement location (a wrist, for example). The blood pressure measurement device 10 measures a blood pressure value of the subject at the measurement location. The blood pressure measurement device 10 includes a blood pressure processing circuit 20. The blood pressure processing circuit 20 calculates the blood pressure representative value on a time period basis, based on the measurement results of the blood pressure value. The blood pressure measurement device 10 can transmit blood pressure information including the measurement results of the blood pressure value and the calculation result of the representative value to the portable terminal 30. The blood pressure processing circuit 20 is an example of the blood pressure management device. The “time period” is an example of a “unit measurement period.”

For example, the portable terminal 30 is a terminal that can be carried by the subject. The portable terminal 30 receives the blood pressure information from the blood pressure measurement device 10. The portable terminal 30 transfers the received blood pressure information to the physician terminal 50 and the server 70. Further, the portable terminal 30 displays diagnostic information received from the physician terminal 50 and the like.

The physician terminal 50 is, for example, a terminal that can be operated by a physician or the like. The physician terminal 50 receives the blood pressure information from the portable terminal 30. The physician terminal 50 generates the diagnostic information related to the subject based on the received blood pressure information and biological information about the subject. The physician terminal 50 transmits the generated diagnostic information to the portable terminal 30 and the server 70.

The server 70 is a server computer that stores information transmitted from the portable terminal 30, the physician terminal 50, and the like.

### 2.2 Hardware Configuration Examples

An example of a hardware configuration of each of the devices of the blood pressure processing system according to the present embodiment is described.

### 2.2.1 Hardware Configuration Example of Blood Pressure Measurement Device

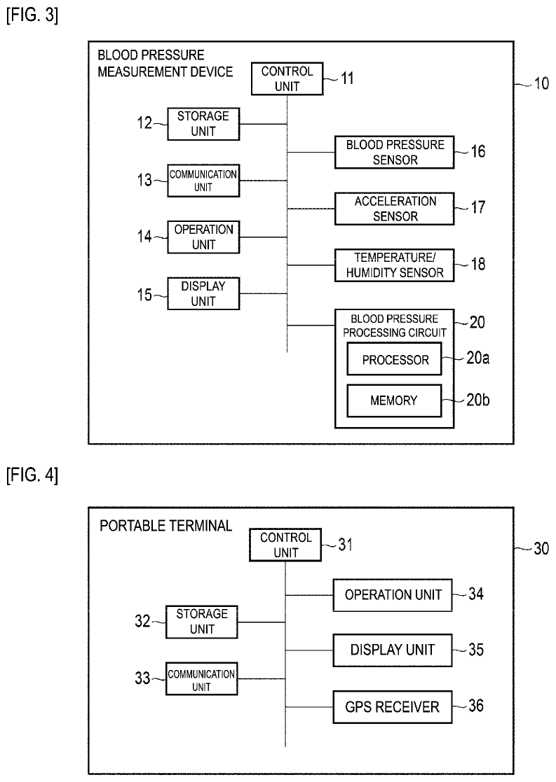

First, a hardware configuration of the blood pressure measurement device 10 according to the present embodiment is described. FIG. 3 is a block diagram illustrating an example of the hardware configuration of the blood pressure measurement device 10 according to the present embodiment. As illustrated in FIG. 3, the blood pressure measurement device 10 according to the present embodiment includes a control unit 11, a storage unit 12, a communication unit 13, an operation unit 14, a display unit 15, a blood pressure sensor 16, and the blood pressure processing circuit 20. The blood pressure measurement device 10 may further include at least one of an acceleration sensor 17 and a temperature/humidity sensor 18.

The blood pressure processing circuit 20 is an example of the blood pressure management device. In the present embodiment, the blood pressure management device is provided in the blood pressure measurement device 10, but the blood pressure management device may be provided in any of the portable terminal 30, the physician terminal 50, or the server 70.

The control unit 11 includes a central processing unit (CPU), a random-access memory (RAM), and a read-only memory (ROM), and controls each component in accordance with information processing. The control unit 11 also to includes a clock (not illustrated) and has a function of acquiring a current date/time. The control unit 11 may have a function of displaying the acquired date/time on the display unit 15.

The control unit 11 generates the blood pressure information, activity information, and environment information based on measurement results obtained by the blood pressure sensor 16, the acceleration sensor 17, and the temperature/humidity sensor 18, and calculation results obtained by the blood pressure processing circuit 20. The blood pressure information includes, for example, a measurement result of the blood pressure value of the subject obtained by the blood pressure sensor 16, and a calculation result of the representative blood pressure value obtained by the blood pressure processing circuit 20. The activity information includes an activity amount, a number of steps, and a sleep condition of the subject, which are based on measurement by the acceleration sensor 17. The environment information includes temperature and humidity around the subject, which are based on measurement by the temperature/humidity sensor 18. Each of the blood pressure information, the activity information, and the environment information is associated with the measurement date/time, which is based on the current date/time acquired by the clock. Further, each of the blood pressure information, and the activity information, and the environment information may further be associated with a device ID for uniquely identifying the blood pressure measurement device 10.

The storage unit 12 is, for example, an auxiliary storage device such as a solid state drive. When the blood pressure measurement device 10 is configured as a device of a certain size rather than as a small device such as a watch, the storage unit 12 may be a hard disk drive. The storage unit 12 stores programs executed by the control unit 11, the blood pressure information, the activity information, the environment information, and the like.

The communication unit 13 is a communication interface for performing communication with the portable terminal 30. For example, the communication unit 13 transmits, to the portable terminal 30, the blood pressure information, the activity information, the environment information, and the like. In the present embodiment, communication with the portable terminal 30 by the communication unit 13 can be achieved by near-field wireless communication such as Bluetooth (registered trademark), but the present embodiment is not limited to this example. For example, the communication performed by the communication unit 13 may adopt communication via the network NW such as a local area network (LAN) or wired communication through use of a communication cable.

For example, the operation unit 14 includes a user interface such as a touch panel and an operation button. The operation unit 14 detects an operation performed by the subject via the user interface, and outputs a signal indicating content of the operation to the control unit 11.

The display unit 15 includes, for example, a display screen (a liquid crystal display (LCD), an electroluminescence (EL) display, or the like), an indicator, and the like. The display unit 15 displays information in accordance with a signal output from the control unit 11, and notifies the subject of the information. For example, the display unit 15 can display the blood pressure information, the activity information, the environment information, and the like stored in the storage unit 12.

The blood pressure sensor 16 measures the blood pressure value of the subject. The blood pressure value includes representative indices such as systolic blood pressure and diastolic blood pressure. In the following description, an example in which the blood pressure value is a value of systolic blood pressure is given, but diastolic blood pressure or another index may be used instead of systolic blood pressure, or a plurality of indices may be used in combination.

The blood pressure sensor 16 may be, for example, a continuous measurement type that is capable of (continuously) measuring blood pressure of the subject per heart beat, or may be a non-continuous measurement type that is capable of measuring blood pressure of the subject in a predetermined time period (discontinuously) on a spot-check basis. For example, a continuous measurement type blood pressure sensor 16 may adopt a method of measuring blood pressure of the subject continuously based on a pulse transit time (PTT), a method of measuring blood pressure of the subject continuously based on a pressure pulse wave (tonometry method), or the like. Note that the method of measuring blood pressure continuously is not limited to the examples described above, and a method of detecting a pulse wave through use of a light emitting element and the like may be adopted as appropriate. For example, the non-continuous measurement type blood pressure sensor 16 may adopt a method of detecting a pulse wave by applying a pressure on a blood vessel through use of a cuff as a pressure sensor (oscillometric method).

The acceleration sensor 17 detects acceleration of the part of the subject on which the blood pressure measurement device 10 is worn, as a set of three axial components. Further, the acceleration sensor 17 may further include a gyro sensor, and may further detect an angular velocity as a set of three axial components in addition to the acceleration.

The temperature/humidity sensor 18 measures the temperature and humidity around the subject.

The blood pressure processing circuit 20 calculates the representative blood pressure value per time period using the blood pressure information generated by the control unit 11 on the basis of the measurement result by the blood pressure sensor 16. The blood pressure processing circuit 20 may be provided in either the blood pressure measurement device 10, the portable terminal 30, the physician terminal 50, or the server 70.

The blood pressure processing circuit 20 includes a processor 20a and a memory 20b, for example. In the blood pressure processing circuit 20, the processor 20a implements various operation controls and data processing by executing programs stored in the memory 20b. Further, the blood pressure processing circuit 20 can include a clock (not illustrated) to count the current date/time.

The processor 20a is, for example, a CPU or a micro processing unit (MPU) that includes an arithmetic circuit or the like. The processor 20a can perform control of each of the units and perform the data processing by executing the programs stored in the memory 20b or the storage unit 12.

The memory 20b includes, for example, a non-volatile memory that stores the programs executed by the processor 20a, and a volatile memory, such as RAM, that is used as a working memory.

The blood pressure processing circuit 20 executes representative value calculation processing on the basis of the blood pressure processing program. The blood pressure processing program is an example of the blood pressure management program. The representative value calculation processing performed by the blood pressure processing circuit 20 is described below. The blood pressure processing program is a program for causing the blood pressure processing circuit 20 to perform the representative value calculation processing. The blood pressure processing program may be stored in the memory 20b or may be stored in the storage unit 12.

Note that the control unit 11 may function as the blood pressure processing circuit 20. In other words, the control unit 11 may also serve as the blood pressure processing circuit 20. In this case, the CPU of the control unit 11 serves as the processor 20a of the blood pressure processing circuit 20, the ROM of the control unit 11 becomes the non-volatile memory of the memory 20b of the blood pressure processing circuit 20, and the RAM of the control unit 11 becomes the volatile memory of the memory 20b of the blood pressure processing circuit 20.

### 2.2.2 Hardware Configuration Example of Portable Terminal

Next, a hardware configuration example of the portable terminal 30 is described. FIG. 4 is a block diagram illustrating an example of the hardware configuration of the portable terminal 30 according to the present embodiment. As illustrated in FIG. 4, the portable terminal 30 according to the present embodiment includes a control unit 31, a storage unit 32, a communication unit 33, an operation unit 34, a display unit 35, and a global positioning system (GPS) receiver 36.

The control unit 31 and the storage unit 32 are similar to the control unit 11 and the storage unit 12 of the blood pressure measurement device 10, respectively. The storage unit 32 of the portable terminal 30 stores information received from the blood pressure measurement device 10 and position information generated by the GPS receiver 36. The information received from the blood pressure measurement device 10 includes the blood pressure information, the activity information, the environment information, and the like.

The communication unit 33 is a communication interface that performs communication with the blood pressure measurement device 10, the physician terminal 50, and the server 70. For example, the communication unit 33 receives the blood pressure information, the activity information, the environment information, and the like from the blood pressure measurement device 10. Further, the communication unit 33 transmits the blood pressure information, the activity information, the environment information, the location information, and the like to the physician terminal 50 and the server 70.

The operation unit 34 and the display unit 35 are similar to the operation unit 14 and the display unit 15 of the blood pressure measurement device 10, respectively.

The GPS receiver 36 measures a position of the portable terminal 30, and generates the position information. For example, the position information includes a positioning date/time and a latitude and a longitude of the portable terminal 30 at the positioning date/time. For example, positioning performed by the GPS receiver 36 may be performed in synchronization with measurement performed by the blood pressure sensor 16 of the blood pressure measurement device 10.

### 2.2.3 Hardware Configuration Example of Physician Terminal

Next, a hardware configuration example of the physician terminal 50 is described. FIG. 5 is a block diagram illustrating an example of the hardware configuration of the physician terminal 50 according to the present embodiment. As illustrated in FIG. 5, the physician terminal 50 according to the present embodiment includes a control unit 51, a storage unit 52, a communication unit 53, an operation unit 54, and a display unit 55.

The control unit 51 and the storage unit 52 are similar to the control unit 11 and the storage unit 12 of the blood pressure measurement device 10, respectively. The control unit 51 of the physician terminal 50 generates the biological information about the subject, the diagnostic information about the subject, and the like.

The storage unit 52 of the physician terminal 50 temporarily stores information transferred from the portable terminal 30, the biological information about the subject, the diagnostic information about the subject, and the like.

The communication unit 53 is a communication interface for performing communication with the portable terminal 30 and the server 70. For example, the communication unit 53 receives, from the portable terminal 30, the blood pressure information, the activity information, the environment information, and the like. Further, the communication unit 53 transmits, to the portable terminal and the server 70, the biological information about the subject, the diagnostic information about the subject, and the like.

The operation unit 54 and the display unit 55 are similar to the operation unit 14 and the display unit 15 of the blood pressure measurement device 10, respectively.

### 2.2.4 Hardware Configuration Example of Server

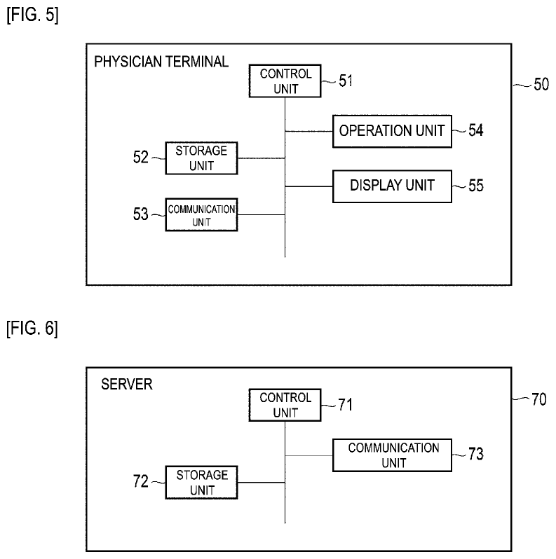

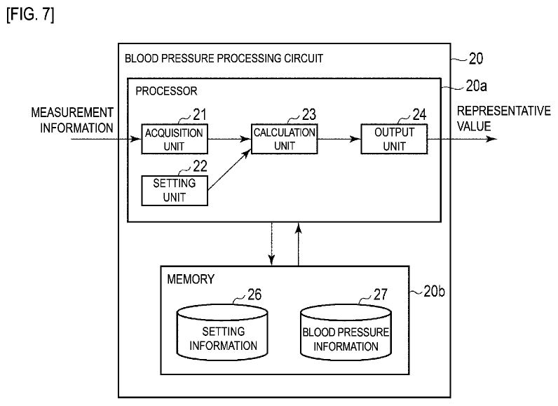

Next, a hardware configuration example of the server 70 is described. FIG. 6 is a block diagram illustrating an example of the hardware configuration of the server 70 according to the present embodiment. As illustrated in FIG. 6, the server 70 according to the present embodiment includes a control unit 71, a storage unit 72, and a communication unit 73.

The control unit 71 and the storage unit 72 are similar to the control unit 11 and the storage unit 12 of the blood pressure measurement device 10, respectively. The storage unit 72 of the server 70 stores information and the like transmitted from the portable terminal 30 and the physician terminal 50.

The communication unit 73 is a communication interface that performs communication with the portable terminal 30 and the physician terminal 50. For example, the communication unit 73 receives, from the portable terminal 30, the blood pressure information, the activity information, the environment information, and the like. For example, the communication unit 73 receives, from the physician terminal 50, the blood pressure information, the activity information, the environment information, and the like. Further, the communication unit 73 receives, from the physician terminal 50, the biological to information about the subject, the diagnostic information about the subject, and the like. The communication unit 73 transmits information stored in the storage unit 72 to the portable terminal 30 and the physician terminal 50.

### 2.3 Functional Configuration Examples

Next, an example of a functional configuration of the blood pressure processing system according to the present embodiment is described.

### 2.3.1 Functional Configuration Example of Blood Pressure Processing Circuit

FIG. 7 is a block diagram schematically illustrating an example of a functional configuration of the blood pressure processing circuit 20 of the blood pressure processing system according to the present embodiment.

The processor 20a of the blood pressure processing circuit 20 loads the blood pressure processing program stored in the non-volatile memory of the memory 20b into the volatile memory of the memory 20b. Then, by interpreting and executing the blood pressure processing program loaded in the volatile memory, the processor 20a functions as an acquisition unit 21, a setting unit 22, a calculation unit 23, and an output unit 24.

A setting information 27, a blood pressure information 26, the representative blood pressure information value, and the like are temporarily stored in the volatile memory of the memory 20b. The blood pressure information 26 includes the measurement value and the measurement date/time of blood pressure, which is the measurement information from the control unit 11, the representative blood pressure value calculated by the calculation unit 23, and the like. The setting information 27 includes information about the unit measurement period set by the setting unit 22, and the like.

The acquisition unit 21 acquires the measurement information, stores the acquired measurement information as the blood pressure information 26 in the memory 20b, and transmits the measurement information to the calculation unit 23.

The setting unit 22 sets the unit measurement period on the basis of the blood pressure fluctuation characteristics. The unit measurement period may be stored in advance in the memory 20b or the storage unit 12, or may be input into the operation unit 14. The unit measurement period includes a plurality of opportunities. The unit measurement period includes, for example, set times for each of time periods. The set times for each of the time periods includes, for example, set times for a morning time period and set times for a night time period. The setting unit 22 can set each of the unit measurement periods by setting switching times of the unit measurement period. The setting unit 22 stores the set switching times in the memory 20b as the setting information 27, and transmits the set switching times to the calculation unit 23.

Based on the blood pressure information 26 and the setting information 27, the calculation unit 23 calculates the representative blood pressure value for each of the time periods. Representative value calculation processing performed by the setting unit 22 and the calculation unit 23 is described below.

The representative blood pressure value in a specific unit measurement period is calculated by, for example, using a plurality of measurement values measured within the specific unit measurement period. The representative blood pressure value in the specific unit measurement period is, for example, an average value or a median value of the plurality of measurement values measured within the specific unit measurement period.

The output unit 24 acquires the representative blood pressure value calculated by the calculation unit 23. The output unit 24 outputs the acquired representative blood pressure value to the storage unit 12 and the like.

### 2.4 Operation Examples

Next, an operation example of the blood pressure processing system according to the present embodiment is described. Note that the processing procedure described below is merely an example, and each process may be changed to the extent possible. Further, in the processing procedure described below, steps can be omitted, substituted, and added in accordance with the embodiment as appropriate.

### 2.4.1 Operation Example of Blood Pressure Processing Circuit

FIG. 8 is a diagram schematically illustrating an example of a procedure for the representative value calculation processing performed by the setting unit 22 and the calculation unit 23 in the blood pressure processing circuit 20 according to the present embodiment.

In the present embodiment, for setting the unit measurement period, the setting unit 22 sets a time period from 00:00 to 12:00 as the morning time period, and sets a time period from 12:00 to 24:00 (00:00) as the night time period. In this case, the setting unit 22 sets 00:00 and 12:00 as the switching times of the time period. By setting a plurality of unit measurement periods, the setting unit 22 divides one day (e.g., 24 hours from a specific time on a chosen day until the same time on the next day) into the plurality of unit measurement periods. The morning time period is an example of a first unit measurement period, for example, and the night time period is an example of a second unit measurement period, for example.

In the representative value calculation processing for the specific unit measurement period, first, the calculation unit 23 enters into a first state M1 at a start time of the unit measurement period. In the first state M1, the calculation unit 23 stands by in the first state M1 until the measurement information is acquired in the acquisition unit 21.

In the first state M1, when the acquisition unit 21 acquires the measurement information from the control unit 11, the processor 20a transitions from the first state M1 to a second state M2. At this time, the calculation unit 23 stores the acquired measurement information in the memory 20b, as first measurement information in the specific time period. The first measurement information is a measurement result obtained by the first measurement in the specific time period. The first measurement information includes a first measurement value and a first measurement time. The first measurement value is a measurement value obtained by the first measurement in the specific time period. The first measurement time is a measurement time of the first measurement in the specific time period.

In the second state M2, when the acquisition unit 21 acquires the measurement information from the control unit 11 within 10 minutes from the first measurement time, the processor 20a transitions from the second state M2 to a third state M3. At this time, the calculation unit 23 stores the acquired measurement information in the memory 20b as second measurement information in the specific time period. The second measurement information is a measurement result obtained by the second measurement in the specific time period. The second measurement information includes a second measurement value and a second measurement time. The second measurement value is a measurement value obtained by the second measurement in the specific time period. The second measurement time is a measurement time of the second measurement in the specific time period. The difference between the first measurement time and the second measurement time is 10 minutes or less. “10 minutes” is an example of “the representative time required for the measurement action of one opportunity.”

In the second state M2, when 10 minutes has elapsed from the first measurement time in a state in which the measurement information is not acquired in the acquisition unit 21, the calculation unit 23 transitions to a fourth state M4.

In the second state M2, when the time becomes 00:00 or 12:00 in the state in which the measurement information is not acquired in the acquisition unit 21, and within 10 minutes from the first measurement time, the calculation unit 23 transitions from the second state M2 to the first state M1. At this time, the calculation unit 23 determines that the time period has switched. “00:00” and “12:00” are examples of a “switching time of the time period.”

For example, in the second state M2, if the time becomes 00:00 in the state in which the measurement information is not acquired in the acquisition unit 21, and within 10 minutes from the first measurement time, the calculation unit 23 determines that the night time period has ended and the morning time period has started. Further, in the second state M2, if the time becomes 12:00 in the state in which the measurement information is not acquired in the acquisition unit 21, and within 10 minutes from the first measurement time, the calculation unit 23 determines that the morning time period has ended and the night time period has started.

In the third state M3, when the acquisition unit 21 acquires the measurement information from the control unit 11 within 10 minutes from the first measurement time, the processor 20a transitions from the third state M3 to a sixth state M6. At this time, the calculation unit 23 stores the acquired measurement information in the memory 20b as third measurement information in the specific time period. The third measurement information is a measurement result obtained by a third measurement in the specific time period. The third measurement information includes a third measurement value and a third measurement time. The third measurement value is a measurement value obtained by the third measurement in the specific time period. The third measurement time is a measurement time of the third to measurement in the specific time period. The difference between the first measurement time and the third measurement time is 10 minutes or less. The calculation unit 23 transitions to the sixth state M6 while the first measurement information, the second measurement information, and the third measurement information in a specific time period are stored in the memory 20b.

In the third state M3, when 10 minutes have elapsed from the first measurement time in the state in which the measurement information is not acquired in the acquisition unit 21, the calculation unit 23 transitions from the third state M3 to the sixth state M6. At this time, the calculation unit 23 transitions to the sixth state M6 with the first measurement information and the second measurement information stored in the memory 20b.

In the third state M3, if the time becomes 00:00 or 12:00 in the state in which the measurement information is not acquired in the acquisition unit 21, and within 10 minutes from the first measurement time, the calculation unit 23 transitions from the third state M3 to a seventh state M7. At this time, the calculation unit 23 determines that the specific time period has switched to the next time period. The calculation unit 23 transitions to the seventh state M7 while the first measurement information and the second measurement information in the specific time period are stored in the memory 20b.

In the fourth state M4, if the time becomes 00:00 or 12:00 in the state in which the measurement information is not acquired in the acquisition unit 21, the calculation unit 23 transitions from the fourth state M4 to the first state M1. At this time, the calculation unit 23 determines that the specific time period has switched to the next time period.

In the fourth state M4, when the acquisition unit 21 acquires the measurement information from the control unit 11, the processor 20a transitions from the fourth state M4 to a fifth state M5. At this time, the calculation unit 23 stores the acquired measurement information in the memory 20b as the second measurement information in the specific time period. The difference between the first measurement time and the second measurement time is 10 minutes or more.

In the fifth state M5, when the acquisition unit 21 acquires the measurement information from the control unit 11 within 10 minutes from the second measurement time, the processor 20a transitions from the fifth state M5 to the sixth state M6. At this time, the calculation unit 23 stores the acquired measurement information in the memory 20b as the third measurement information in the specific time period. The difference between the second measurement time and the third measurement time is 10 minutes or less. The calculation unit 23 transitions to the sixth state M6 while the first measurement information, the second measurement information, and the third measurement information in the specific time period are stored in the memory 20b.

In the fifth state M5, when 10 minutes have elapsed from the second measurement time in the state in which the measurement information is not acquired in the acquisition unit 21, the calculation unit 23 transitions from the fifth state M5 to the sixth state M6. The calculation unit 23 transitions to the sixth state M6 while the first measurement information and the second measurement information in the specific time period are stored in the memory 20b.

In the fifth state M5, if the time becomes 00:00 or 12:00 in the state in which the measurement information is not acquired in the acquisition unit 21, and within 10 minutes from the second measurement time, the calculation unit 23 transitions from the fifth state M5 to the seventh state M7. At this time, the calculation unit 23 determines that the specific time period has switched to the next time period. The calculation unit 23 transitions to the seventh state M7 while the first measurement information and the second measurement information in the specific time period are stored in the memory 20b.

In the sixth state M6, the calculation unit 23 uses the measurement information stored in the memory 20b to calculate the representative blood pressure value in the specific time period. The calculation unit 23 stores the calculated representative value in the memory 20b, and transmits the calculated representative value to the output unit 24.

In the sixth state M6, if the time becomes 00:00 or 12:00, the calculation unit 23 determines that the specific time period has switched to the next time period, and transitions from the sixth state M6 to the first state M1. In other words, after calculating the representative value in the sixth state M6, even if the measurement information is acquired in the acquisition unit 21, that measurement information is not used for the calculation of the representative value.

In the seventh state M7, the calculation unit 23 uses the measurement information stored in the memory 20b to calculate the representative blood pressure value in the specific time period. The calculation unit 23 stores the calculated representative value in the memory 20b, and transmits the calculated representative value to the output unit 24. The calculation of the representative blood pressure value in the seventh state M7 is performed in the same manner as the calculation of the representative blood pressure value in the sixth state M6, for example. In the seventh state M7, the calculation unit 23 transitions to the first state M1 immediately after the calculation of the representative value has ended.

### 2.5 Actions and Effects

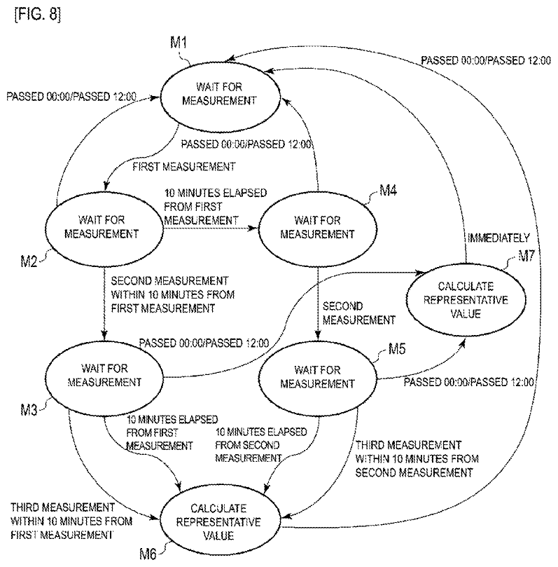

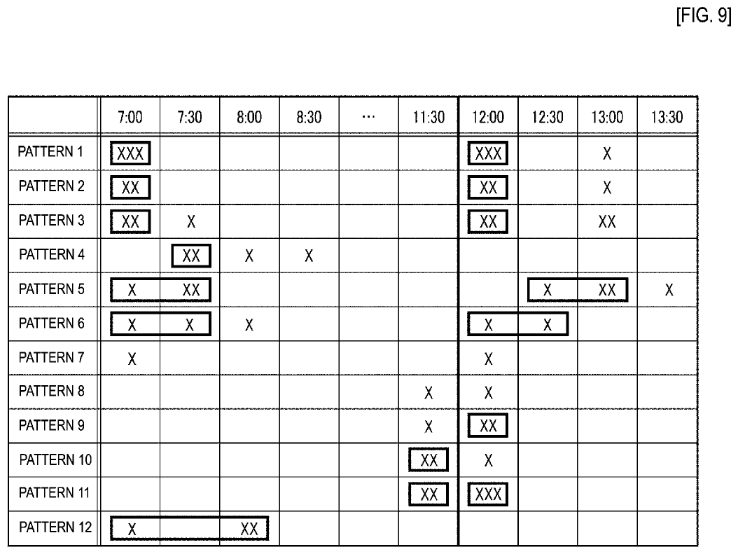

Effects of the present embodiment are described below with reference to FIG. 9. FIG. 9 illustrates an example of the measurement information employed in the representative value calculation processing performed by the blood pressure processing circuit 20 according to the present embodiment.

In the example illustrated in FIG. 9, the fact that a blood pressure measurement has been performed is indicated by a X mark. When a plurality of X marks are illustrated adjacent to each other, this indicates that a plurality of measurements have been performed within 10 minutes. “Within 10 minutes” is an example of “within a time period of one opportunity.” One opportunity is, for example, a time period typically required to perform one action. The one opportunity is, for example, 10 minutes.

For example, a pattern 1 indicates that three measurements have been performed within 10 minutes in a time period from 07:00 to 07:30. Further, when the X marks are not adjacent to each other, this indicates that corresponding measurement times are separated by 10 minutes or more. Note that, in the example illustrated in FIG. 9, it is assumed that no measurement is performed before 07:00 and after 14:00.

Further, in the example illustrated in FIG. 9, of the measurement results, the measurement results used for calculating the representative blood pressure value are surrounded by bold lines. For example, in the pattern 1, four blood pressure measurements are performed in the night time period, and the measurement results of the first to third measurements are used for calculating the representative blood pressure value and are reflected in the representative blood pressure value.

For example, in a pattern 7, one blood pressure measurement is performed in the morning time period. The first measurement is performed in the time period from 07:00 to 07:30. In this case, the calculation unit 23 transitions in order from the first state M1 to the second state M2 and the first state M1. Therefore, in the morning time period of the pattern 7, the calculation unit 23 does not transition to the sixth state M6 or the seventh state M7, and thus the calculation of the representative value is not performed.

As described above, it is assumed that the present embodiment is used in a region in which it is recommended to use two or more measurement results to calculate the representative value in the specific unit measurement period. According to the present embodiment, if only a single blood pressure measurement is performed in a specific time period, the calculation of the representative blood pressure value is not performed. Therefore, the calculation of the representative blood pressure value using the number of measurement results that does not satisfy the prescribed number is prevented. As a result, a representative value satisfying the prescribed number of measurements can be obtained as the blood pressure value.

Further, for example, in the pattern 1, four blood pressure measurements are performed in the night time period. The first to third measurements are performed in a time period from 12:00 to 12:30. The first to third measurements are performed within 10 minutes. The fourth measurement is performed in a time period from 13:00 to 13:30. The fourth measurement is performed after 10 minutes or more have elapsed from the first measurement.

In the pattern 1, in the night time period, the calculation unit 23 transitions in order from the first state M1 to the second state M2, the third state M3, and the sixth state M6. For calculation of the representative value in the sixth state M6, the measurement results obtained by the first to third measurements are reflected in the representative blood pressure value for the morning time period. Further, when the third measurement is performed, the calculation unit 23 starts calculating the representative blood pressure value. Thus, even if the fourth measurement is performed in the sixth state M6, that measurement value is not reflected in the representative blood pressure value.

As described above, it is assumed that the present embodiment is used in a region in which it is recommended to use two or more measurement results to calculate the representative value in the specific unit measurement period. Thus, in the calculation of the representative blood pressure value for the specific time period, it is sufficient that three measurement results be reflected. According to the present embodiment, when four or more blood pressure measurements are performed in the specific time period, measurement results obtained by the fourth and subsequent measurements are not reflected in the representative value. As a result, the representative blood pressure value can be efficiently calculated.

Further, for example, in a pattern 2, three blood pressure measurements are performed in the night time period. The first and second measurements are performed in the time period from 12:00 to 12:30. The first and second measurements are performed within 10 minutes. The third measurement is performed in the time period from 13:00 to 13:30. The third measurement is performed after 10 minutes or more have elapsed from the second measurement.

In the pattern 2, in the night time period, the calculation unit 23 transitions in order from the first state M1 to the second state M2, the third state M3, and the sixth state M6. For calculation of the representative value in the sixth state M6, the measurement results obtained by the first and second measurements are reflected in the representative blood pressure value for the night time period. Further, when 10 minutes have elapsed from the first measurement, the calculation unit 23 starts calculating the representative blood pressure value. Thus, even if the third measurement is performed in the sixth state M6, that measurement value is not reflected in the representative blood pressure value.

As described above, it is assumed that the present embodiment is used in a region in which it is recommended to use two or more measurement results to calculate the representative value in a specific unit measurement period. Meanwhile, measurement results obtained after 10 minutes or more have elapsed from the first measurement may not be suitable for calculating the representative value. According to the present embodiment, when two or more measurement values have been obtained in the specific time period, and then, measurement is performed after 10 minutes or more have elapsed from the first measurement, the measurement result from the measurement performed after 10 minutes or more have elapsed from the first measurement is not reflected in the representative value. As a result, an appropriate representative value that satisfies the prescribed number of measurements and in which unnecessary measurement results are not reflected can be obtained as the blood pressure value.

Further, for example, in a pattern 6, two blood pressure measurements are performed in the night time period. The first measurement is performed in the time period from 12:00 to 12:30. The second measurement is performed in a time period from 12:30 to 13:00. The second measurement is performed after 10 minutes or more have elapsed from the first measurement.

In the pattern 6, in the night time period, the calculation unit 23 transitions in order from the first state M1 to the second state M2, the fourth state M4, the fifth state M5, and the sixth state M6. In the sixth state M6, the measurement results obtained by the first and second measurements are reflected in the representative blood pressure value for the night time period.

Further, for example, in the pattern 6, three blood pressure measurements are performed in the morning time period. The first measurement is performed in the time period from 07:00 to 07:30. The second measurement is performed in a time period from 07:30 to 08:00. The second measurement is performed after 10 minutes or more have elapsed from the first measurement. The third measurement is performed in a time period from 08:00 to 08:30. The third measurement is performed after 10 minutes or more have elapsed from the second measurement.

In the pattern 6, in the morning time period, the calculation unit 23 transitions in order from the first state M1 to the second state M2, the fourth state M4, the fifth state M5, and the sixth state M6. In the sixth state M6, the measurement results obtained by the first and second measurements are reflected in the representative blood pressure value for the morning time period. Further, when 10 minutes have elapsed from the second measurement, the calculation unit 23 starts calculating the representative blood pressure value. Thus, even if the third measurement is performed in the sixth state M6, that measurement value is not reflected in the representative blood pressure value.

Here, in the unit measurement period, by performing another operation between the first measurement and the second measurement, the second measurement may be performed after 10 minutes or more have elapsed from the first measurement. In this case, the first measurement result and the second measurement result are useful for calculating the representative value, since both the measurements are performed in the unit measurement period. However, because the second measurement is not performed in the same opportunity as the first measurement, even though the first measurement result and the second measurement result are useful for calculating the representative blood pressure value, the representative blood pressure value in which the first measurement result and the second measurement result are reflected may not be calculated.

According to the present embodiment, when the second measurement is performed in the specific time period, even when the second measurement is performed after 10 minutes or more have elapsed from the first measurement, the representative blood pressure value is calculated using the first measurement result and the second measurement result. Thus, even when the second measurement is performed after 10 minutes or more have elapsed from the first measurement, a representative blood pressure value that satisfies the prescribed number and in which useful measurement results are appropriately reflected can be obtained.

Further, for example, in a pattern 5, three blood pressure measurements are performed in the morning time period. The first measurement is performed in the time period from 07:00 to 07:30. The second and third measurements are performed in the time period from 07:30 to 08:00. The second measurement is performed after 10 minutes or more have elapsed from the first measurement. The second and third measurements are performed within 10 minutes of each other.

In the pattern 5, in the morning time period, the calculation unit 23 transitions in order from the first state M1 to the second state M2, the fourth state M4, the fifth state M5, and the sixth state M6. In the sixth state M6, the measurement results obtained by the first to third measurements are reflected in the representative blood pressure value for the morning time period.

Further, for example, in the pattern 5, four blood pressure measurements are performed in the night time period. The first measurement is performed in the time period from 12:30 to 13:00. The second and third measurements are performed in the time period from 13:00 to 13:30. The second measurement is performed after 10 minutes or more have elapsed from the first measurement. The second and third measurements are performed within 10 minutes of each other. The fourth measurement is performed in a time period from 13:30 to 14:00. The fourth measurement is performed after 10 minutes or more have elapsed from the third measurement.

In the pattern 5, in the night time period, the calculation unit 23 transitions in order from the first state M1 to the second state M2, the fourth state M4, the fifth state M5, and the sixth state M6. In the sixth state M6, the measurement results obtained by the first to third measurements are reflected in the representative blood pressure value for the night time period. Further, when the third measurement is performed, the calculation unit 23 starts calculating the representative blood pressure value. Thus, even if the fourth measurement is performed in the sixth state M6, that measurement value is not reflected in the representative blood pressure value.

Here, when measuring blood pressure, by performing another operation between the first measurement and the second measurement, the second measurement may be performed after 10 minutes or more have elapsed from the first measurement, and further, the third measurement may be performed within 10 minutes from the second measurement. In this case, because the second measurement is not performed in the same opportunity as the first measurement, even though the measurement results obtained by the first to third measurements are useful for calculating the representative blood pressure value, the measurement results obtained by the first to third measurements may not be appropriately reflected in the representative blood pressure value.

According to the present embodiment, when the second measurement is performed after 10 minutes or more have elapsed from the first measurement and the third measurement is performed within 10 minutes from the second measurement in the specific time period, the representative blood pressure value is calculated using the measurement results obtained by the first to third measurements. Thus, a representative blood pressure value in which useful measurement results are more appropriately reflected can be obtained.

Here, when the subject has a lifestyle as indicated by the pattern 1, for example, the subject measures their blood pressure before going to bed in the time period from 07:00 to 07:30 and measures their blood pressure after waking up in the time period from 12:00 to 12:30. In this case, the measurement results obtained by the measurements performed in the time period from 12:00 to 12:30 are useful for calculating the representative blood to pressure value, as the blood pressure values measured after waking up.

In the present embodiment, the time period from 00:00 to 12:00 is set as the morning time period, and the time period from 12:00 to 24:00 is set as the night time period. Thus, the measurement results obtained by the measurements performed in the time period from 07:00 to 07:30 are reflected in the calculation of the representative value of the first unit measurement period, and the measurement results obtained by the measurements performed in the time period from 12:00 to 12:30 are reflected in the calculation of the representative value of the second unit measurement period. Therefore, even when the subject has the lifestyle as indicated by the pattern 1, useful values such as the blood pressure values after waking up are reflected in the representative blood pressure value.

In this way, in the present embodiment, a time that belongs to neither the morning time period nor the night time period is not provided. In other words, there is no time of the day that does not correspond to any of the unit measurement periods. As a result, all times of the day are included in one of the unit measurement periods. Thus, according to the present embodiment, even if the subject is a shift worker whose living hours are changeable or is a person who has a lifestyle spanning over a plurality of standard times, the representative blood pressure value in which useful measurement results are appropriately reflected can be calculated.

Further, in the present embodiment, the calculation unit 23 transitions to the third state M3, based on the acquisition of the second measurement information in the second state M2. Further, the calculation unit 23 transitions to the fifth state M5, based on the acquisition of the second measurement information in the fourth state M4. Then, in the third state M3 and the fifth state M5, the calculation unit 23 stands by for the acquisition of the third measurement information. Then, in the third state M3 and the fifth state M5, regardless of whether or not the third measurement information has been acquired, the calculation unit 23 transitions to either the sixth state M6 or the seventh state M7 within 10 minutes from the second measurement time. Thus, the state in which the calculation unit 23 stands by for the acquisition of the third measurement information ends within 10 minutes after the acquisition time of the second measurement information.

On the other hand, the calculation unit 23 transitions to the second state M2, based on the acquisition of the first measurement information in the first state M1. Then, in the second state M2, the calculation unit 23 stands by for the acquisition of the second measurement information. Then, in the second state M2, when the second measurement information is not acquired, the calculation unit 23 stands by for the acquisition of the second measurement information until the switching time of the time period is reached. Thus, the state in which the calculation unit 23 stands by for the acquisition of the second measurement information may be maintained, for example, for several hours after the acquisition time of the first measurement information. Therefore, the maximum time from the acquisition time of the first measurement information to the end of the stand-by state for the acquisition of the second measurement information is longer than the maximum time from the acquisition time of the second measurement information to the end of the stand-by state for the acquisition of the third measurement information.

### 2.6 First Modified Example of First Embodiment

A first modified example of the first embodiment is described below. Note that, in the following, the same reference numerals are used for components that are the same as those of the above-described embodiment, and descriptions thereof are omitted as appropriate.

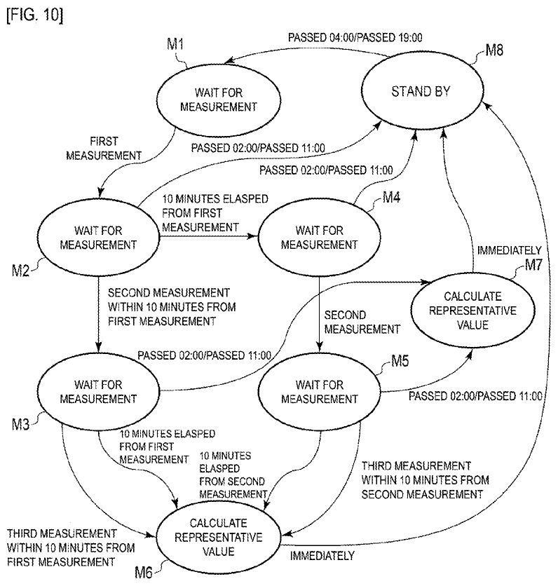

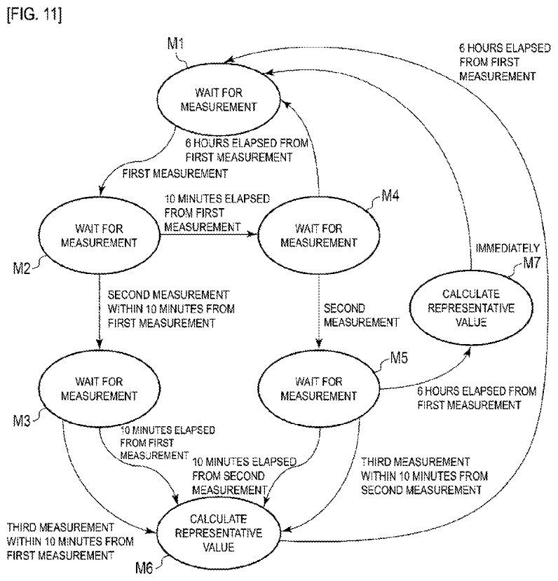

FIG. 10 is a diagram schematically illustrating an example of the procedure for the representative value calculation processing performed by the setting unit 22 and the calculation unit 23 in the blood pressure processing circuit 20 according to the present modified example.

In the present modified example, in the setting of the unit measurement period, the setting unit 22 sets a time period from 04:00 to 11:00 as the morning time period, and sets a time period from 19:00 to 02:00 as the night time period. The setting unit 22 sets 04:00 as a start time of the morning time period, and sets 11:00 as an end time of the morning time period. Further, the setting unit 22 sets 19:00 as a start time of the night time period, and sets 02:00 as an end time of the night time period. Then, the setting unit 22 sets 04:00, 11:00, 19:00, and 02:00 as the time period switching times. The morning time period is an example of the first unit measurement period, for example, and the night time period is an example of the second unit measurement period, for example.

In the representative value calculation processing for a specific unit measurement period according to the present modified example, in the second state M2, if the time becomes 02:00 or 11:00 in the state in which the measurement information is not acquired in the acquisition unit 21, and within 10 minutes from the first measurement time, the calculation unit 23 transitions from the second state M2 to an eighth state M8. At this time, the calculation unit 23 determines that the morning time period or the night time period has ended.

In the third state M3, if the time becomes 02:00 or 11:00 in the state in which the measurement information is not acquired in the acquisition unit 21, and within 10 minutes from the first measurement time, the calculation unit 23 transitions from the third state M3 to the seventh state M7. At this time, the calculation unit 23 determines that the morning time period or the night time period has started. The calculation unit 23 transitions to the seventh state M7 while the first measurement information and the second measurement information in the specific time period are stored in the memory 20b.

In the fourth state M4, if the time becomes 02:00 or 11:00 in the state in which the measurement information is not acquired in the acquisition unit 21, the calculation unit 23 transitions from the fourth state M4 to the eighth state M8. At this time, the calculation unit 23 determines that the morning time period or the night time period has ended.

In the fifth state M5, if the time becomes 02:00 or 11:00 in the state in which the measurement information is not acquired in the acquisition unit 21, and within 10 minutes from the second measurement time, the calculation unit 23 transitions from the fifth state M5 to the seventh state M7. At this time, the calculation unit 23 determines that the morning time period or the night time period has ended. The calculation unit 23 transitions to the seventh state M7 while the first measurement information and the second measurement information in the specific time period are stored in the memory 20b.

In the sixth state M6 or the seventh state M7, the calculation unit 23 transitions to the eighth state M8 immediately after the calculation of the representative value has ended.

In the eighth state M8, the calculation unit 23 stands by until the time becomes 04:00 or 19:00. When the time becomes 04:00 or 19:00, the calculation unit 23 transitions from the eighth state M8 to the first state M1. At this time, the calculation unit 23 determines that the morning time period or the night time period has started.

In the present modified example, the end time of the night time period is set to 02:00. Thus, even when the blood pressure measurement for the night time period is performed in a time period from 00:00 to 02:00 on the next day, that measurement information is used as the measurement value of the night time period for calculating the representative blood pressure value for the night time period continued from the previous day. As a result, for example, even to when the subject performs the blood pressure measurement before going to bed in the night time period, in the time period from 0:00 to 02:00, the measurement information is appropriately reflected in the representative value. As in the present modified example, by setting the unit measurement period appropriately according to the lifestyle, the culture, and the like of the subject, it is possible to obtain the representative blood pressure value that is more accurate.

### 2.7 Second Modified Example of First Embodiment

A second modified example of the first embodiment is described below. Note that, in the following, the same reference numerals are used for components that are the same as those of the above-described embodiment, and descriptions thereof are omitted as appropriate.

FIG. 11 is a diagram schematically illustrating an example of the procedure for the representative value calculation processing performed by the setting unit 22 and the calculation unit 23 in the blood pressure processing circuit 20 according to the present modified example.

In the present modified example, in the setting of the unit measurement period, the setting unit 22 sets the switching time of the time period based on the measurement information. The setting unit 22 sets a time that is six hours after the first measurement in a specific time period, as an end time of the specific time period. Based on the arrival of the end time of the specific time period, the calculation unit 23 determines that the specific time period has ended and the time has switched to the next time period. The specific time period is an example of the first unit measurement period, for example, and the next time period is an example of the second unit measurement period.

In the representative value calculation processing for the specific unit measurement period according to the present modified example, in the fourth state M4, if six hours have elapsed from the first measurement time in the state in which the measurement information is not acquired in the acquisition unit 21, the calculation unit 23 transitions from the fourth state M4 to the first state M1. At this time, the calculation unit 23 determines that the specific time period has ended and the time has switched to the next time period.

In the fifth state M5, if six hours have elapsed from the first measurement time in the state where the measurement information is not acquired in the acquisition unit 21, and within 10 minutes from the second measurement time, the calculation unit 23 transitions from the fifth state M5 to the seventh state M7. At this time, the calculation unit 23 determines that the time period has switched. The calculation unit 23 transitions to the seventh state M7 while the first measurement information and the second measurement information in the specific time period are stored in the memory 20b.

In the sixth state M6 or the seventh state M7, if six hours have elapsed from the first measurement time, the calculation unit 23 transitions to the first state M1. At this time, the calculation unit 23 determines that the time period has switched.

If the switching time of the time period is set to a certain time of day, when the lifestyle of the subject changes depending on the day, an appropriate representative value may not be calculated. For example, when a time period from 05:00 to 14:00 is set as the morning time period, when the subject measures their blood pressure from 06:00 before going to bed and measures their blood pressure from 13:00 after waking up, both the measurement result from 06:00 and the measurement result from 13:00 are processed as the measurement results of the morning time period. In this case, the measurement result from 13:00 may not be reflected in the representative value as the measurement result after waking up.

In the present modified example, when a predetermined time period (six hours in this case) has elapsed from the first measurement, it is determined that the time period has switched. Thus, for example, when the blood pressure measurement is performed from 06:00 before going to bed, and the blood pressure measurement is performed from 13:00 after waking up, 12:00, which is six hours after the first measurement, is set as the switching time of the time period, and the measurement result from 06:00 is reflected in the representative blood pressure value in the specific time period. Then, the measurement result from 13:00 is reflected in the representative blood pressure value for the next time period.

In this way, according to the present modified example, by setting the unit measurement period based on the measurement information, even when the lifestyle of the subject is not constant, the representative blood pressure value can be calculated in which useful measurement results, such as a measurement value obtained when waking up, are appropriately reflected.

### 2.8 Third Modified Example of First Embodiment

A third modified example of the first embodiment is described below. Note that, in the following, the same reference numerals are used for components that are the same as those of the above-described embodiment, and descriptions thereof are omitted as appropriate.

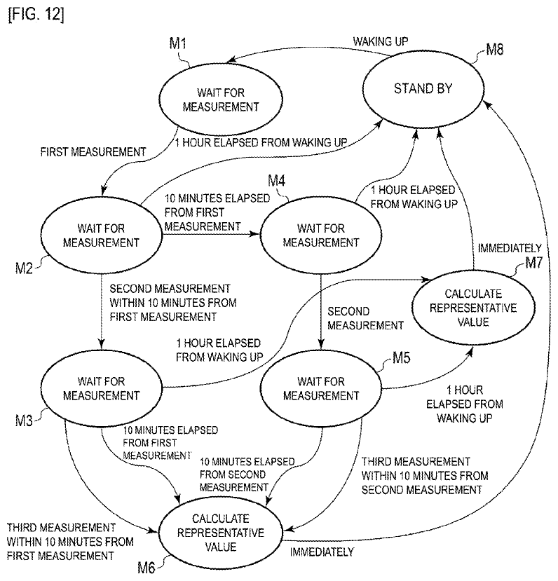

FIG. 12 is a diagram schematically illustrating an example of the procedure for the representative value calculation processing performed by the setting unit 22 and the calculation unit 23 in the blood pressure processing circuit 20 according to the present modified example.

In the present modified example, the acquisition unit 21 acquires sleep information of the subject in addition to the measurement information of the subject. For example, the acquisition unit 21 can acquire a sleep state from the control unit 11 and obtain the sleep information based on the sleep state. The sleep state is detected, for example, by the control unit 11 based on measurement results from the acceleration sensor 17 or other additional sensors. Further, the sleep information may be generated in a simplified manner based on an activated state and an inactivated state of a wakeup application of the portable terminal 30, or the like. The sleep information includes, for example, a sleep start time and a wake-up time (sleep end time) of the subject.

For setting of the unit measurement period, the setting unit 22 sets the switching time of the time period based on the sleep information. The setting unit 22 sets a time that is one hour after the wake-up time as an end time of a specific time period. Based on the arrival of the end time of the specific time period, the calculation unit 23 determines that the specific time period has ended. The specific time period is an example of the first unit measurement period.

In the representative value calculation processing for a specific unit measurement period, according to the present modified example, in the second state M2, if one hour has elapsed from the wake-up time in the state in which the measurement information is not acquired in the acquisition unit 21, and within 10 minutes from the first measurement time, the calculation unit 23 transitions from the second state M2 to the eighth state M8. At this time, the calculation unit 23 determines that the morning time period has ended.

In the third state M3, if one hour has elapsed from the wake-up time in the state in which the measurement information is not acquired in the acquisition unit 21, and within 10 minutes from the first measurement time, the calculation unit 23 transitions from the third state M3 to the seventh state M7. At this time, the calculation unit 23 determines that the morning time period has ended. The calculation unit 23 transitions to the seventh state M7 while to the first measurement information and the second measurement information in the specific time period are stored in the memory 20b.

In the fourth state M4, if one hour has elapsed from the wake-up time in the state in which the measurement information is not acquired in the acquisition unit 21, the calculation unit 23 transitions from the fourth state M4 to the eighth state M8. At this time, the calculation unit 23 determines that the morning time period has ended.

In the fifth state M5, if one hour has elapsed from the wake-up time in the state in which the measurement information is not acquired in the acquisition unit 21, and within 10 minutes from the second measurement time, the calculation unit 23 transitions from the fifth state M5 to the seventh state M7. At this time, the calculation unit 23 determines that the morning time period has ended. The calculation unit 23 transitions to the seventh state M7 while the first measurement information and the second measurement information in the specific time period are stored in the memory 20b.

In the sixth state M6 or the seventh state M7, the calculation unit 23 transitions to the eighth state M8 immediately after the calculation of the representative value. At this time, the calculation unit 23 determines that the morning time period has ended.

In the eighth state M8, the calculation unit 23 determines whether or not the subject has woken up based on the sleep information acquired in the acquisition unit 21. In the eighth state M8, when it is determined that the subject has woken up, the calculation unit 23 transitions from the eighth state M8 to the first state M1.

In the present modified example, the wake-up time is acquired, and based on the wake-up time, it is determined that the specific unit measurement period has ended. Thus, according to the present modified example, by setting the unit measurement period based on the wake-up time, even when the lifestyle of the subject is not constant, useful measurement results such as the measurement value obtained after waking up can be easily identified. As a result, a representative blood pressure value can be calculated in which useful measurement results, such as the measurement value obtained after waking up, are more appropriately reflected.

Note that in the present modified example also, the second unit measurement period may be provided in addition to the first unit measurement period. In this case, for example, a time that is six hours after the wake-up time is set as a start time of the second unit measurement period, and the sleep start time is set as an end time of the second unit measurement period. Alternatively, a time that is six hours after the first measurement after waking up may be set as the start time of the second unit measurement period.

Further, the first embodiment and each of the modified examples may be freely combined. For example, by combining the third modified example and the first modified example, a time period from the wake-up time to 12:00 may be set as the first unit measurement period, and a time period that starts after 12 hours have elapsed from the wake-up time may be set as the second unit measurement period.

### 3. Second Embodiment

A second embodiment of the blood pressure management device according to the application example described above is described below. In the following, a blood pressure processing system including a blood pressure measurement device is described as an example of the blood pressure management device. Note that, in the following, the same reference numerals are used for components that are the same as those of the first embodiment, and descriptions thereof are omitted as appropriate.

The blood pressure processing system according to the present embodiment is a system that calculates the representative blood pressure value for each of the unit measurement periods using blood pressure values measured from the subject, and that stores or presents the calculated representative value. It is assumed that the blood pressure processing system according to the present embodiment is used in a region in which it is recommended to use one to three measurement results to calculate the representative value in a specific unit measurement period. It is assumed that the blood pressure processing system according to the present embodiment is used in Japan, for example.

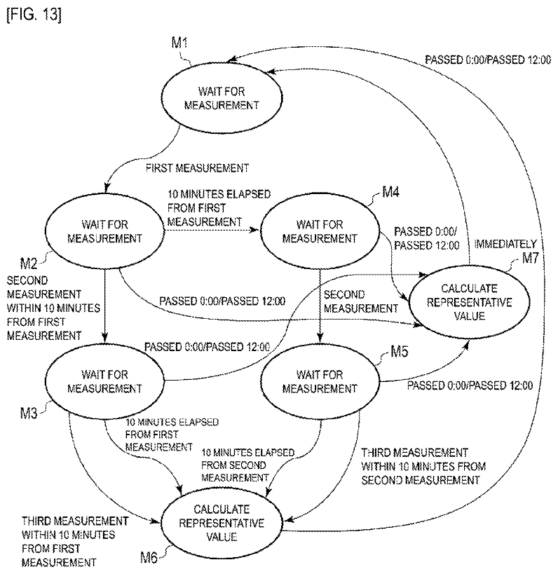

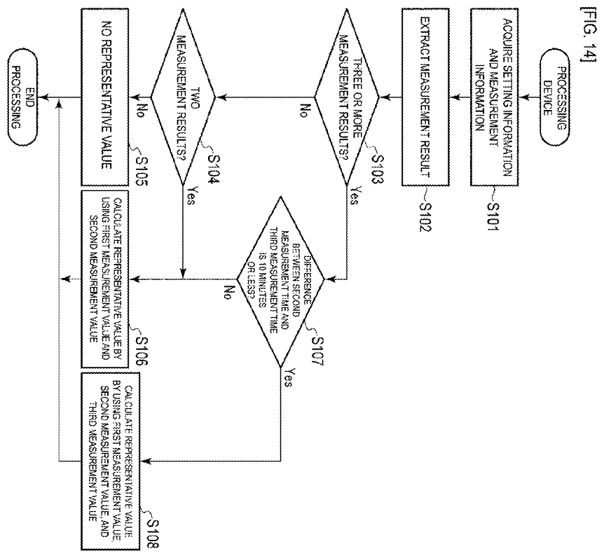

FIG. 13 is a diagram schematically illustrating an example of the procedure for the representative value calculation processing performed by the setting unit 22 and the calculation unit 23 in the blood pressure processing circuit 20 according to the present embodiment.

In the present embodiment, in the second state M2, if the time becomes 00:00 or 12:00 in the state in which the measurement information is not acquired in the acquisition unit 21, and within 10 minutes from the first measurement time, the calculation unit 23 transitions from the second state M2 to the seventh state M7. At this time, the calculation unit 23 determines that the time period has switched.

In the fourth state M4, if the time becomes 00:00 or 12:00 in the state in which the measurement information is not acquired in the acquisition unit 21, the calculation unit 23 transitions from the fourth state M4 to the seventh state M7. At this time, the calculation unit 23 determines that the time period has switched.

For example, in the pattern 7, which is an example illustrated in FIG. 9, one blood pressure measurement is performed in the morning time period. The first measurement is performed in the time period from 07:00 to 07:30. In this case, the calculation unit 23 transitions between the states in the order of the first state M1, the second state M2, and the seventh state M7. For calculation of the representative value in the seventh state M7, the measurement result of the first measurement is reflected in the representative blood pressure value for the morning time period.

As described above, it is assumed that the present embodiment is used in a region in which it is recommended to use at least one measurement result to calculate the representative value in the specific unit measurement period. According to the present embodiment, the calculation of the representative blood pressure value is performed even when only one blood pressure measurement is performed in the specific unit measurement period. As a result, a representative value conforming to the stipulation regarding the prescribed number of measurements can be obtained as the blood pressure value.

It is needless to say that in the present embodiment also, the unit measurement period can be set in the same manner as described in each of the modified examples of the first embodiment described above.

### 4. Third Embodiment

A third embodiment of the blood pressure management device according to the application example described above is described below. In the following, a blood pressure processing system including a blood pressure measurement device is described as an example of the blood pressure management device. Note that, in the following, the same reference numerals are used for components that are the same as those of the first embodiment, and descriptions thereof are omitted as appropriate.

In the present embodiment, the processor 20a uses stored to measurement results obtained in the past to calculate the representative blood pressure value for a specific unit measurement period. For example, the processor 20a starts the representative value calculation processing for the specific unit measurement period based on transmission of an instruction to start the representative value calculation processing. At this time, the start instruction for calculation of the representative value is transmitted from the control unit 11, for example, based on an operation that is input using the operation unit 14. The specific unit measurement period is input, for example, to the operation unit 14.

Alternatively, the processor 20a may periodically perform the representative value calculation processing. The representative value calculation processing performed by the processor 20a is performed using once a day as an interval, for example. In this case, the processor 20a acquires the measurement information for one day, from the previous representative value calculation processing up to the present, and performs the representative value calculation processing for each of the unit measurement periods within this time period. The representative value calculation processing may be performed using once a week as the interval, for example.

FIG. 14 is a flowchart illustrating an example of the procedure for the representative value calculation processing in the blood pressure processing circuit 20 according to the present embodiment.

As illustrated in FIG. 14, in the representative value calculation processing for a specific unit measurement period, first, the processor 20a obtains the setting information and the measurement information (S101). The acquired information is stored in the memory 20b, for example. The measurement information includes at least one measurement result. The measurement result includes a measurement value and a measurement time. The setting information includes a start time and an end time of the specific unit measurement period.

Next, the processor 20a extracts the measurement results within the specific unit measurement period based on the measurement time of each of the measurement results and the setting information for the specific unit measurement period (S102). At this time, the processor 20a determines whether or not each of the measurement results is a measurement result in the specific unit measurement period, by determining whether or not the measurement time is included within the specific unit measurement period for each of the measurement results.

Next, the processor 20a determines whether or not the number of measurement results in the specific unit measurement period is three or more (S103). In this way, the processor 20a determines whether or not the number of measurements in the unit measurement period is three or more.

When the number of measurement results in the specific unit measurement period is two or less (No at S103), the processor 20a determines that the number of measurements in the unit measurement period is two or less. In this case, the processor 20a determines whether or not the number of measurement results in the specific unit measurement period is two (S104).

When the number of measurement results in the specific unit measurement period is not two (No at S104), the processor 20a determines that the number of measurements in the unit measurement period is one or zero. In this case, the processor 20a determines that the number of measurements in the unit measurement period does not satisfy the prescribed number, and does not calculate the representative blood pressure value (S105).

When the number of measurement results in the specific unit measurement period is two (Yes at S104), the processor 20a determines that the number of measurements in the unit measurement period is two. In this to case, based on the measurement times, the processor 20a identifies whether each of the measurement results in the specific unit measurement period is the first measurement result or the second measurement result. The first measurement result includes the first measurement value and the first measurement time, and the second measurement result includes the second measurement value and the second measurement time. Then, the processor 20a uses the first measurement value and the second measurement value to calculate the representative blood pressure value (S106).

When the number of measurement results in the specific unit measurement period is three or more (Yes at S103), the processor 20a determines that the number of measurements in the unit measurement period is three or more. In this case, based on the measurement times, the processor 20a identifies the first measurement result, the second measurement result, and the third measurement result among each of the measurement results in the specific unit measurement period. The first measurement result includes the first measurement value and the first measurement time, and the second measurement result includes the second measurement value and the second measurement time. Further, the third measurement result includes the third measurement value and the third measurement time.

Next, the processor 20a determines whether or not the difference between the second measurement time and the third measurement time is 10 minutes or less (S107). “10 minutes” is an example of “one opportunity.”

When the difference between the second measurement time and the third measurement time is greater than 10 minutes (No at S107), the processor 20a determines that the second measurement and the third measurement are not performed in the same opportunity. In this case, the processor 20a uses the first measurement value and the second measurement value to calculate to the representative blood pressure value (S106).

When the difference between the second measurement time and the third measurement time is 10 minutes or less (Yes at S107), the processor 20a determines that the second measurement and the third measurement are performed in the same opportunity. In this case, the processor 20a uses the first measurement value, the second measurement value, and the third measurement value to calculate the representative blood pressure value (S108).

The processor 20a performs the above-described representative value calculation processing, for example, for all of the unit measurement periods included in a time period from the previous representative value calculation processing to the current representative value calculation processing.

In the present embodiment, the representative value calculation processing performed by the blood pressure processing circuit 20 is performed by reading the past measurement information. Thus, according to the present embodiment, the subject can know the representative blood pressure value in the specific unit measurement period at any timing.

### 5. Common Configurations Between Embodiments

A blood pressure management device (1, 20) includes an acquisition unit (2, 21) configured to acquire a measurement value obtained by measuring blood pressure, a setting unit (3, 22) configured to set a unit measurement period including a plurality of opportunities based on blood pressure fluctuation characteristics, and a calculation unit (4, 23) configured to calculate a representative blood pressure value for the unit measurement period based on the measurement value. When a second measurement is performed in a different opportunity from that of a first measurement within the unit measurement period, the calculation unit (4, 23) calculates the representative value for the unit measurement period by using both a first measurement value obtained by the first measurement and a second measurement value obtained by the second measurement.

Note that the present invention is not limited to the embodiment, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

Supplementary Notes

A part or the entirety of each of the embodiments described above can be described as in the following supplementary notes in addition to the scope of the claims, but the present invention is not limited thereto.

(Supplementary Note 1)

A blood pressure management device comprising:

a hardware processor; and
a memory, wherein
the hardware processor acquires a measurement value obtained by a blood pressure measurement using a sensor and causes the memory to store the measurement value,
the hardware processor causes the memory to store a setting of a unit measurement period that is set based on blood pressure fluctuation characteristics and that includes a plurality of opportunities, and
when a second measurement is performed in a different opportunity from a first measurement within the unit measurement period stored in the memory, the hardware processor calculates a representative blood pressure value for the unit measurement period by using both a first measurement value obtained by the first measurement and stored in the memory, and a second measurement value obtained by the second measurement and stored in the memory.

(Supplementary Note 2)

A blood pressure management method executed by a device including a hardware processor and a memory, the blood pressure management method comprising:

a step of the hardware processor acquiring a measurement value obtained through blood pressure measurement using a sensor and causing the memory to store the measurement value;
a step of the hardware processor causing the memory to store a setting of a unit measurement period that is set based on blood pressure fluctuation characteristics and includes a plurality of opportunities; and
a step of, when a second measurement is performed in a different opportunity from a first measurement within the unit measurement period stored in the memory, the hardware processor calculating a representative blood pressure value for the unit measurement period by using both a first measurement value obtained by the first measurement and stored in the memory, and a second measurement value obtained by the second measurement and stored in the memory.

### REFERENCE SIGNS LIST

1 Blood pressure management device
2 Acquisition unit
3 Setting unit
4 Calculation unit
5 Output unit
6 Blood pressure measurement device
11 Control unit
12 Storage unit
13 Communication unit
14 Operation unit
15 Display unit
16 Blood pressure sensor
17 Acceleration sensor
18 Temperature/humidity sensor
20 Blood pressure processing circuit
20a Processor
20b Memory
21 Acquisition unit
22 Setting unit
23 Calculation unit
24 Output unit
26 Blood pressure information
27 Setting information
30 Portable terminal
31 Control unit
32 Storage unit
33 Communication unit
34 Operation unit
35 Display unit
36 GPS receiver
50 Physician terminal
51 Control unit
52 Storage unit
53 Communication unit
54 Operation unit
55 Display unit
70 Server
71 Control unit
72 Storage unit
73 Communication unit

1. A blood pressure management device comprising:
an acquisition unit configured to acquire a measurement value obtained by measurement of blood pressure;
a setting unit configured to set, based on blood pressure fluctuation characteristics, a unit measurement period including a plurality of opportunities; and
a calculation unit configured to calculate, based on the measurement value, a representative value of the blood pressure for the unit measurement period, wherein
when an opportunity in which the first measurement is performed and an opportunity in which the second measurement is performed are different within the unit measurement period, the calculation unit calculates the representative value for the unit measurement period by using both a first measurement value obtained by the first measurement and a second measurement value obtained by the second measurement, and
when the opportunity in which the first measurement is performed and the opportunity in which the second measurement is performed are different within the unit measurement period, and a third measurement is performed in a different opportunity from the opportunity in which the second measurement is performed within the unit measurement period, the calculation unit calculates the representative value by using only the first measurement value and the second measurement value.

2. The blood pressure management device according to claim 1, wherein
the calculation unit determines whether an opportunity in which the first measurement is performed and an opportunity in which the second measurement is performed are identical based on a representative time period required for a measurement action in one opportunity.

3. The blood pressure management device according to claim 2, wherein
when the first measurement and the second measurement are performed within a time period of not more than 10 minutes, the calculation unit determines that the opportunity in which the first measurement is performed and the opportunity in which the second measurement is performed are identical.

4. The blood pressure management device according to claim 1, wherein
when the opportunity in which the first measurement is performed and the opportunity in which the second measurement is performed are identical within the unit measurement period, and a third measurement is performed in a different opportunity from the opportunity in which the first measurement is performed within the unit measurement period, the calculation unit calculates the representative value for the unit measurement period by using only the first measurement value and the second measurement value.

5. The blood pressure management device according to claim 1, wherein
when the opportunity in which the first measurement is performed and the opportunity in which the second measurement is performed are different within the unit measurement period, and a third measurement is performed in an opportunity identical to the opportunity in which the second measurement is performed within the unit measurement period, the calculation unit calculates the representative value by using the first measurement value, the second measurement value, and a third measurement value obtained by the third measurement.

6. The blood pressure management device according to claim 1, wherein
the calculation unit does not use measurement values obtained by fourth and subsequent measurements within the unit measurement period for the calculation of the representative value.

7. The blood pressure management device according to claim 1, wherein
the setting unit sets a plurality of the unit measurement periods to divide one day into the plurality of unit measurement periods.

8. The blood pressure management device according to claim 1, wherein
the setting unit sets an end time of the unit measurement period based on a measurement time of the first measurement.

9. The blood pressure management device according to claim 1, wherein
the setting unit sets a time period of not more than 15 hours as the unit measurement period.

10. The blood pressure management device according to claim 1, wherein
the acquisition unit further acquires a wake-up time, and
the setting unit sets the wake-up time as a start time of the unit measurement period, and, based on the wake-up time, sets an end time of the unit measurement period.

11. The blood pressure management device according to claim 1, wherein
in the calculation of the representative value by the calculation unit, a maximum time from acquisition of the first measurement value until an end of a stand-by state for acquisition of the second measurement value is longer than a maximum time from acquisition of the second measurement value until an end of a stand-by state for acquisition of a third measurement value obtained by a third measurement within the unit measurement period.

12. The blood pressure management device according to claim 1, further comprising:
a blood pressure sensor; and
a display unit.

13. A non-transitory computer-readable storage medium storing a blood pressure management program for causing a processor to execute a function of each of units included in the blood pressure management device according to claim 1.

14. A blood pressure management method comprising:
acquiring a measurement value obtained by measurement of blood pressure;
setting, based on blood pressure fluctuation characteristics, a unit measurement period including a plurality of opportunities; and
calculating, based on the measurement value, a representative value of the blood pressure for the unit measurement period, wherein
when an opportunity in which a second measurement is performed and an opportunity in which a first measurement is performed are different within the unit measurement period, the representative value for the unit measurement period is calculated by using both a first measurement value obtained by the first measurement and a second measurement value obtained by the second measurement, and
when the opportunity in which the first measurement is performed and the opportunity in which the second measurement is performed are different within the unit measurement period, and a third measurement is performed in a different opportunity from the opportunity in which the second measurement is performed within the unit measurement period, the representative value is calculated by using only the first measurement value and the second measurement value.

15. A blood pressure management device comprising:
an acquisition unit configured to acquire a measurement value obtained by measurement of blood pressure;
a setting unit configured to set, based on blood pressure fluctuation characteristics, a unit measurement period including a plurality of opportunities; and
a calculation unit configured to calculate, based on the measurement value, a representative value of the blood pressure for the unit measurement period, wherein
when an opportunity in which the first measurement is performed and an opportunity in which the second measurement is performed are different within the unit measurement period, the calculation unit calculates the representative value for the unit measurement period by using both a first measurement value obtained by the first measurement and a second measurement value obtained by the second measurement, and
in the calculation of the representative value performed by the calculation unit, a maximum time from acquisition of the first measurement value until an end of a stand-by state for acquisition of the second measurement value is longer than a maximum time from the acquisition of the second measurement value until an end of a stand-by state for acquisition of a third measurement value obtained by a third measurement within the unit measurement period.

16. The blood pressure management device according to claim 15, wherein
the calculation unit determines whether the opportunity in which the first measurement is performed and the opportunity in which the second measurement is performed are identical based on a representative time period required for a measurement action of one opportunity.

17. The blood pressure management device according to claim 16, wherein
when the first measurement and the second measurement are performed within 10 minutes within the unit measurement time, the calculation unit determines that the opportunity in which the first measurement is performed and the opportunity in which the second measurement is performed are identical.

18. The blood pressure management device according to claim 15, wherein
when the opportunity in which the second measurement is performed and the opportunity in which the first measurement is performed are identical within the unit measurement period, and a third measurement is performed in a different opportunity from the opportunity in which the first measurement is performed within the unit measurement period, the calculation unit calculates the representative value for the unit measurement period by using only the first measurement value and the second measurement value.

19. The blood pressure management device according to claim 15, wherein
when the opportunity in which the second measurement is performed and the opportunity in which the first measurement is performed are different within the unit measurement period, and a third measurement is performed in a different opportunity from the opportunity in which the second measurement is performed within the unit measurement period, the calculation unit calculates the representative value by using only the first measurement value and the second measurement value.

20. The blood pressure management device according to claim 15, wherein
when the opportunity in which the second measurement is performed and the opportunity in which the first measurement is performed are different within the unit measurement period, and a third measurement is performed in an opportunity identical to the opportunity in which the second measurement is performed within the unit measurement period, the calculation unit calculates the representative value by using the first measurement value, the second measurement value, and a third measurement value obtained by the third measurement.

21. The blood pressure management device according to claim 15, wherein
the calculation unit does not use measurement values obtained by fourth and subsequent measurements within the unit measurement period for the calculation of the representative value.

22. The blood pressure management device according to claim 15, wherein
the setting unit sets a plurality of the unit measurement periods to divide one day into the plurality of unit measurement periods.

23. The blood pressure management device according to claim 15, wherein
the setting unit sets an end time of the unit measurement period based on a measurement time of the first measurement.

24. The blood pressure management device according to claim 15, wherein
the setting unit sets a time period of not more than 15 hours as the unit measurement period.

25. The blood pressure management device according to claim 15, wherein
the acquisition unit further acquires a wake-up time, and
the setting unit sets the wake-up time as a start time of the unit measurement period, and, based on the wake-up time, sets an end time of the unit measurement period.

26. The blood pressure management device according to claim 15, further comprising:
a blood pressure sensor; and
a display unit.

27. A non-transitory computer-readable storage medium storing a blood pressure management program for causing a processor to execute functions of each of units included in the blood pressure management device according to claim 15.

28. A blood pressure management method comprising:
acquiring a measurement value obtained by measurement of blood pressure;
setting, based on blood pressure fluctuation characteristics, a unit measurement period including a plurality of opportunities; and
calculating, based on the measurement value, a representative value of the blood pressure for the unit measurement period, wherein
when an opportunity in which a second measurement is performed and an opportunity in which a first measurement is performed are different within the unit measurement period, the representative value for the unit measurement period is calculated by using a first measurement value obtained by the first measurement and a second measurement value obtained by the to second measurement, and a maximum time from acquisition of the first measurement value until an end of a stand-by state for acquisition of the second measurement value is longer than a maximum time from the acquisition of the second measurement value until an end of a stand-by state for acquisition of a third measurement value obtained by a third measurement within the unit measurement period.
